(19) United States
(12) Patent Application Publication
(10) Pub. No.: US 2006/0121472 A1
(43) Pub. Date: Jun. 8, 2006

(54) Method for determining the allelic state of the 5'-end of the $g(a)s1- casein gene

(76) Inventors: Eva-Maria Prinzenberg, Giessen (DE); George Erhardt, Pohlheim (DE)

Correspondence Address: CLARK & BRODY, 1090 VERMONT AVENUE, NW, SUITE 250, WASHINGTON, DC, 20005, US

(21) Appl. No.: 10/524,295

(22) PCT Filed: Aug. 15, 2003
(86) PCT No.: PCT/DE03/02747

§ 371 Date: Sep. 29, 2005

(30) Foreign Application Priority Data
Aug. 16, 2002 (DE) 102 38 433.9

(51) Int. Cl. C12Q 1/68; C07H 21/04

(52) U.S. Cl. 435/6; 536/23.5

(57) ABSTRACT

The invention refers to a genetic marker at the 5′-flanking region of the alpha-S1 casein gene (CSN1S1) and the casein gene complex as well as a method to classify cattle, independent of age and lactation, through determination of the allelic state within this area as well as the application of this method to select organisms with a preferred allele, for instance in the marker-supported selection.

The invention refers to a genetic marker at the 5′-flanking region of the αS1 casein gene (CSN1S1) and the casein gene complex as well as a method to classify cattle, independent of age and lactation, through determination of the allelic state within this area as well as the application of this method to select organisms with a preferred allele, for instance in the marker-supported selection.

### TECHNICAL STATE OF THE ART

The hereditary potential of breed animals (regarding the milk protein content and other characteristics relevant to the breeding) is estimated at present through estimating the breeding value based on test matings and performance records of the descendants. The disadvantage of this conventional procedure is obvious. For cattle, it takes approx. 3 years from the first insemination by a test bull until the first daughters begin lactating, thus approx. 4 years until the registration of a complete lactation of the daughter. Only thereafter can the breed value be estimated. Until then, from both maintaining the bulls until the first estimated breeding values are available and the test mating, costs arise, which are substantial during this long period and due to the total amount of animals. This applies analogically to the registration of the own contribution and the determination of a breed value of cows.

Therefore, for some years, with the help of the progress in the genome analysis, international efforts have been undertaken to develop genetic markers and direct gene tests to identify performance parameters relevant to breeding. Hereby, genome-wide analysis of markers have made possible, with the help of the linkage analysis, the approximation to the chromosome range, in which the gene locations are situated, which are relevant to the determination of performance, the so-called QTL regions (QTL=quantitative trait loci). Such QTL studies and the resulting tests are described among others in the WO 20000 36143 and the WO 2001 57250 A2/A3. Further details of the QTL analysis of farm animals as well as a procedure based on QTL studies to also isolate causal candidate genes, are described in DE 100 17 675 A1. The disclosure of the invention is included therein.

Regarding cattle and other species bred for milk production, the crucial criteria are the milk quantity, the protein content and fat. For these criteria, different QTL were identified, among other locations on the chromosome BTA 6. The potential QTL regions for protein contents are indicated relatively uniformly from different working groups within the area around or between the micro satellite markers BM143 and TGLA37 and thus approximately 20-30 centimorgans (cM) away from the casein locus (Spelman et al. 1996, Genetics 144, 1799-1808; Georges et al. 1995, Genetics 139, 907-920; Boldly et al. 1996, J Anim Breed Genet 133, 355-362; Zhang et al. 1998, Genetics 149, 1959-1973). According to Nadesalingam et al. (2001, Mammalian Genome 12, 27-31) the casein genes are, however, as well excluded as candidates for the observed QTL effects due to their position (40 cM away from the QTL).

Since the mid 80's, genetically conditioned milk protein varieties of cattle have been analysed with regard to an influence on milk yield and quality characteristics. This was realized partly by registering (in the milk) the phenotipically distinguishable protein variants (Ng-Kwai-Hang et al., 1984, J Dairy Sci 67, 835-840 and Ng-Kwai-Hang et al., 1986, J Dairy Sci 69, 22-26,), later by means of molecular genetic processes, which provided evidence of the genetic mutations upon which the protein variants are based (Sabour et al., 1996, J Dairy Sci 79, 1050-1056). Studies up to now partly reveal conflicting results of the examined variants which can not always be confirmed for different breeds and regional origins (summarised by Prinzenberg, 1998, ISBN 3-922306-68-3, chapter 2.4, p. 14-21). The majority of these studies are concentrated on variants of the β-lactoglobulin, and the β- and κ-casein, since in the αs1 casein, the frequency of only two protein variants is worth mentioning and in particular, in the already strongly selected milk breeds like Holstein Friesian/German Holstein, the protein variant αs1 casein B can be almost exclusively found (Ng-Kwai-Hang et al., 1990, J Dairy Sci 73, 3414-3420; Erhardt et al., 1993, J Animal Breed Genet 36, 145-152; Lien et al., 1999, Animal Genetics 30, 85-91). In a more recent study of dairy cattle with different proportions of Holstein blood (Freyer et al., 1999, J Animal Breed Genet 116, 87-97), αs1 casein was also not used in the linkage analysis due to the lack of variability.

Various tests are described concerning the molecular genetic differentiation of the αs1 casein variants B and C (David & Deutch 1992, Animal Genetics 23, 425-429; Schlee & Rottmann 1992, J Anim Breed Genet 109, 316-319). Individual gene test procedures for the rare alleles A, D and F also exist, (Prinzenberg 1998, ISBN 3-922306-68-3; chapter 4.1, p. 61-71), as well as for the proof of a quantitative variant of the αs1 casein G (Mariani et al 1995, L'industria del Latte 31, 3-13). By means of sequencing around 1,000 base pairs (bp) from the 5′-region of the αs1 casein gene from various cattle breeds, Schild & Geldermann (1996) showed 17 variable positions in the 5′-flanking region of the CSN1S1 gene, of which 5 have been detected due to different recognition sequences for the restriction endonucleases with Polymerase Chain Reaction-Restriction Fragment Length Polymorphism (PCR-RFLP). According to Ehrmann et al. (1997, J Animal Breed Genet 114, 121-132), the 5′-flanking variants are each linked with certain protein alleles, in such a manner that the existence of given protein variants implies the existence of certain variants in the 5′-flanking region. Koczan et al. (1993, Animal Genetics 24, 74) also described a gene test to discriminate the αs1 casein B against C in German American Holstein, Black and White, and Jersey cows which is based on a fragment from the 5′-flanking region of the αs1 casein. For the last test mentioned, the strict linkage with the protein mutations αs1 casein B and C has however in the meantime been refuted and therewith, the validity for the following breeds: Aberdeen Angus, Anatolian black, Angeln, Asturian Valley, Ayrshire, British Frisian, Casta Navarra, Charolais, Chianina, Fighting Bull, Hereford, Jersey, Maremmana, Pezzata Rossa, Piedmontese, Scottish Highland, Turkish Grey Steppe (Jann et al., 2001; Arch. Tierz, Dummerstorf 45, 13-21).

The casein genes are mapped as a closely linked gene locus in cattle and sheep at chromosome 6, in humans in chromosome 4, and in mice at chromosome 5. The linking of casein genes has also been proven for other animal species (rabbit, pig, goat). Due to this close linkage, the present designation of the site of the αs1 casein gene in the genetic map of cattle is linked to the site of the κ-casein gene. In the up-to-date gene map for cattle, the physical position BTA6q31-33, and the genetic position 82.6 cM (MARC97) and 103.0 cM (IBRP97) resp., are stated for both genes. For this reason, the recombination rate between the αs1 casein and κ-casein gene is assumed to be zero.

The utilization of a lactalbumin sequence for the selection of breeding animals is revealed in EP0555435. Likewise, for the bovine κ-casein, there exist numerous gene tests (Denicourt et al., 1990, Animal Genetics 21, 215-216; Medrano & Aguilar-Cordova, 1990, Biotechnology 8, 144-145; Pinder et al., 1991, Animal Genetics 22, 11-20; Schlee & Rottmann, 1992, J Animal Breed. Genet. 109, 153-155; Zadworny & Kuhnlein, 1990, Theor. Appl. Genet. 80, 631-634), since influences on the processing characteristics and cheese producing ability of the milk are attributed to this protein (see Lodes et al., 1996, Milchwissenschaft 51, 368-373 and 543-548).

Due to the mammary gland-specific expression, the promoters of bovine milk protein genes and also the αs1 casein gene are utilized in the creation of transgenic animals and for expression in cell cultures. DE 38 54 555 T2, the content of which is referred to here, describes the utilization of the αs1 casein promoter and signal peptide for the production of recombinant proteins in the milk of mammals. Rudolf also gives an overview of the use of transgenic animals for the production of recombinant proteins and the promoters used for this purpose. (1999, Trends in Biotechnology (TIBTECH) 17, 367-374).

Winter et al. (2002, PNAS 99, 9300-9305) describe a direct gene test for a gene from the fatty acid metabolism (DGAT1) and attribute an effect on the milk fat content to this gene.

The disadvantage of all procedures which phenotypically differentiate (namely within milk samples) the genetic variants, based on a milk sample, lies in the fact that lactating cows can be studied exclusively. Thus, there exists the need to be able to examine the animal independent of lactation. Furthermore, the proven polymorphisms in the region of the milk protein gene to be coded do not represent a reliable marker for milk performance traits according to the current state of the art. The existing QTL analyses point to a QTL 1 which lies outside the milk protein genes. For αs1 casein, there is currently no marker available with sufficient variability, causing the fact that this gene's effects on lactating performance and content characteristics can almost not be studied. All available test procedures depend on the molecular genetic differentiation of the also phenotypically available variation.

The micro satellite markers, which was ascertained through QTL analyses, are only suited for conditional use in the marker protected selection, because the respective marker-QTL-linkage must first be explained. In each case, with these microsatellite markers it is a matter of indirect tests which, depending on the closeness of the linkage to the causal gene location, have less reliable results. The disadvantage of the procedures in EP 0555435 lies in the fact that α-lactalbumin only makes up a small portion (ca. 2-5%) of the entire milk protein. At approx. 80%, the caseins (αs1-, αs2-, β- and κ-casein) form the largest portion of the total protein. Thus, by applying these selection markers, only minor breeding progress is to be expected.

The gene test for DGAT1 from Winter et al. has the disadvantage that, from the perspective of the breeder and milk producer, the milk fat content is not of primary interest, but rather takes second place behind the protein content.

The disadvantage of the procedures in DE 38 54 555 T2 lies in the fact that the utilized portion of the αs1 casein promoter is not more closely characterized by means of a nucleotide sequence. A 9 kb fragment is utilized containing exons I and II, terminated with recognition sites for KpnI and BamHI. No consideration of the exact base effect or possible variations takes place which can influence the effectiveness of the expression with this segment of the promoter.

Currently, there are no reliable markers for milk protein content and no direct genetic test for a functional gene segment in order to test an animal's genetic potential, independent of age and lactation.

### PROBLEMS OF THE INVENTION

Hence, it is the problem of the invention to make available a genetic marker and a procedure for the classification of milk production traits in order to examine an animal's milk production traits by means of their genetic material, independently of age and lactation.

This problem is solved by making available, within the region of the αs1 casein gene, a marker which remains polymorphic and genetic also within selected milk breeds, and through a procedure which enables the classification of the animals independently of age and lactation, the genetic mapping of the αs1 casein gene, the examination of effects which are either closely linked with this gene location or thereby directly caused, as well as a breeding utilization.

Based on the invention, the procedure refers to a genetic test for a functional gene segment, the reliability of the results is greater than with linkage markers and the test result is available within a few days to a few hours, whereby the substantial costs of test mating can be reduced. Thus, the procedure based on the invention eliminates the described disadvantages in the technical state of the art.

With the marker based on the invention the selection of especially advantageous promoters for the production of expression vectors and transgenic animals is possible.

In an especially advantageous practical embodiment, the invention consists of a test kit, which contains the oligonucleotides for the enrichment of a segment of the marker sequence of the αs1 casein gene, preferably the primer 1 CSN1S1pro1f (5′ GAA TGA ATG AAC TAG TTA CC 3′), primer 2 CSN1S1pro1r (5′ GAA GAA GCA GCA AGC TGG 3′) and primer 3 CSN1S1pro2r (5′ CCT TGA AAT ATT CTA CCA G 3′) as well as reference probes for one or more sequences of the marker sequence of the αs1 casein gene and alleles thereof.

The following figures are enclosed with the description:

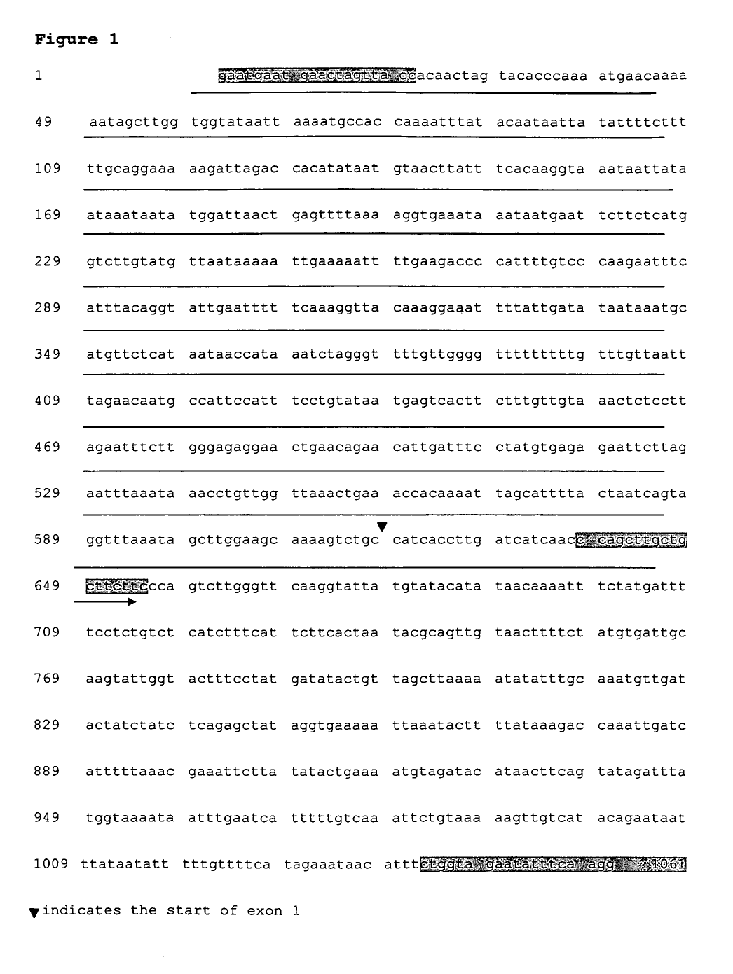

FIG. 1 DNA-sequence from the 5′-flanking region of the αs1 casein gene, in the following designated as marker sequence

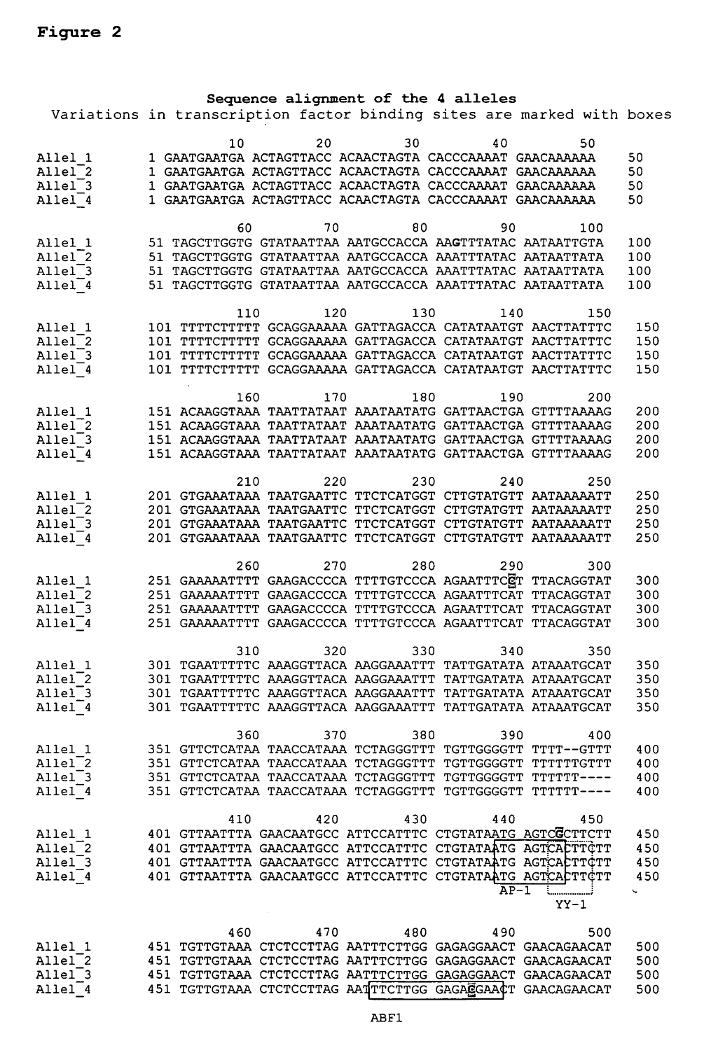

FIG. 2 Alignment of the nucleic acid sequences of the allelic state of the αs1 casein gene allele 1, allele 2, allele 3, allele 4 (differences in potential transcription factor-interfaces are highlighted)

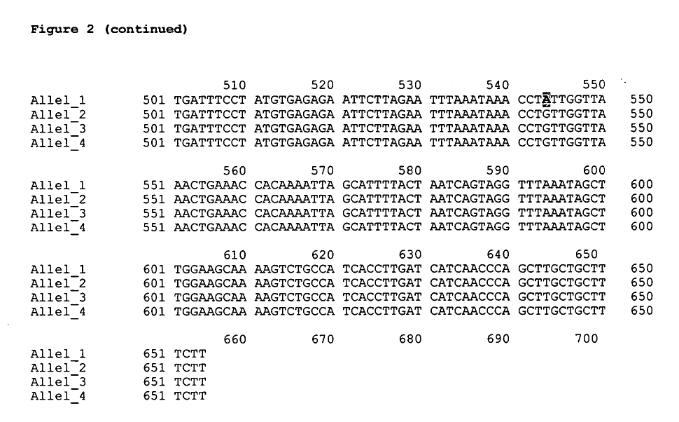

FIG. 3 Schematic representation of the migration pattern of the alleles 1 to 4 of the marker CSN1S1 in the analysis SSCP.

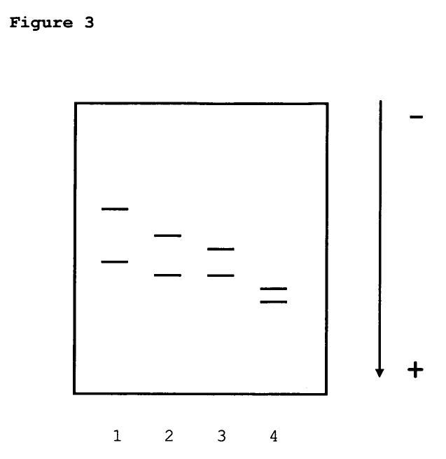

FIG. 4 Result of the variance analysis

It was surprisingly discovered that the examined sequence segment, which is flanked by the oligonucleotides CSN1S1pro1f and CSN1S1pro1r or CSN1S1pro2r (grey box in FIG. 1) within the breed German Holstein, contains four alleles which were detectable through a single-strand conformation polymorphism analysis and thus is sufficiently polymorphic in order to realize a genetic mapping and analysis concerning the effects of the alleles on the milk performance parameters.

This concerns a fragment of 1061 bp within the 5′-flanking region and the exon 1 (refer to FIG. 1), in particular the fragment of 654 bp which is flanked by the two oligonucleotides CSn1S1pro1f and CSN1S1pro1r.

The four alleles were cloned and sequenced. The sequence analysis was in accordance with the sequence published by Koczan et al. (1991, Nucleic Acids Research 19, 5591-56596; Genbank Acc. No. X59856) for allele 2, except for the length of poly-T (from position 390 of FIG. 1 onwards). The alleles 1, 3 and 4 differ from this sequence by various substitutions and deletions. The variable positions are highlighted in the sequence alignment (FIG. 2). In alleles 1 and 4, potential transcription factor-binding sites are each affected by mutations. Thus, in allele 1, two potential binding sites (for AP-1 and YY1) cease to exist, whereas in allele 4, a new potential ABF1-binding site emerges.

The polymorphism found is therefore located in a supposedly functional gene region and thus, is a suitable marker for milk production traits, in particular for the protein content.

Based on the current invention, the sequence fragment is flanked by the following oligonucleotide sequence, which is utilized as a primer for amplification by means of PCR, whereby the combinations Primer 1 with Primer 2, and Primer 1 with Primer 3 are possible:

Primer 1:
CSN1S1pro1f (5′ GAA TGA ATG AAC TAG TTA CC 3′)
Primer 2:
CSN1S1pro1r (5′ GAA GAA GCA GCA AGC TGG 3′)
Primer 3:
CSN1S1pro2r (5′ CCT TGA AAT ATT CTA CCA G 3′)

The primer binding sites are shaded grey in FIG. 1.

A procedure, based on the invention, is made available, which can be carried out directly at the hereditary material of the organism to be examined. With the help of the marker based on the current invention, a genetic mapping of the αs1 casein gene within the linkage map is made possible and the determination of the allelic condition in individual organisms, e.g. cattle, is undertaken, which determines within a few hours the genetic potential with regard to milk protein content.

The procedure for determining the genetic potential with regard to milk protein content by determining the allelic condition of the marker based on the current invention, in detail, consists of:

1. Making available the genetic material of the organism to be examined, from male or female breeding cattle or an embryo thereof.

The organism is, by definition, an animal, particularly a mammal, in particular a bovine, a sheep or a goat, including embryos of these species.

The organism is also a genetically modified organism (GMO), which contains the described sequence fragment of the 5′-flanking region (FIG. 1) and of the αs1 casein gene or parts thereof.

The genetic material is, by definition, genomic DNA or RNA from animals, but also plasmid DNA from bacteria, from artificial chromosomes such as BACs and YACs or constructions created from genetic material of various organisms for specific applications, e.g. for the production of transgenic organisms.

The source material for the extraction of material containing DNA or RNA is namely blood, leukocytes, tissue including biopsy material, milk, sperm, hair, several cells including cell material from embryos, a bacteria culture or isolated chromosomes. Furthermore, genetic material already amplified beforehand, which contains the marker sequence (FIG. 1) or parts thereof, is again source material.

2. Selective isolation or enrichment of the sequence fragments of FIG. 1, or a sequence which contains portions thereof, preferably the illustrated sequence fragment from position 1 to 654 of FIG. 1.

The isolation of genetic material is achieved by standard methods as they are described in the handbook “Molecular Cloning” (Sambrook, Fritsch, Maniatis, 1989; Cold Spring Harbour Laboratory Press, New York), or can be carried out by using commercially obtainable kits (e.g. Nucleospin, Machery Nagel, Düren, Deutschland).

The enrichment is achieved preferably by means of polymerase chain-reaction (PCR, Mullis & Falloona, 1987, Methods in Enzymology 155, 335-350), whereby fluorescently marked, radioactively marked, or chemically marked primers can also be utilized. When using RNA as genetic material, a reverse transcription must be carried out beforehand (Myers & Gelfand 1991, Biochemistry 30, 7661-7666).

The sequence fragment is enhanced preferably by the following oligonucleotide sequences based on the current invention, which are utilized as a primer for the amplification by means of PCR, whereby the combinations primer 1 with primer 2, and primer 1 with primer 3 are possible

The sequence fragment is enhanced preferably with the following oligonucleotide sequences, based on the current invention, as primer for the amplification, whereby the combinations Primer 1 with Primer 2 and Primer 1 with Primer 3 are possible:

Primer 1:
CSN1S1pro1f (5′ GAA TGA ATG AAC TAG TTA CC 3′)
Primer 2:
CSN1S1pro1r (5′ GAA GAA GCA GCA AGC TGG 3′)
Primer 3:
CSN1S1pro2r (5′ CCT TGA AAT ATT CTA CCA G 3′)

The selection of further primers is explicitly possible, which makes possible the amplification of a partial sequence of the sequence described in FIG. 1, within which variable nucleotide positions are located in order to differentiate the alleles 1 to 4.

3. Proof of the allelic state in the isolated or enhanced sequence fragment of FIG. 1, preferably within the partial sequence, which is flanked by CSN1S1pro1f and CSn1S1pro1r.

In order to determine the allelic state, various standard techniques are available which are well known by the expert: The sequencing according to Sanger et al. 1977, through an illustration of single-strand conformation polymorphisms (SSCP, Orita et al. 1989, Genomics 5, 874-879), restriction fragment length polymorphisms (RFLP; Botstein et al. 1980, American Journal of Human Genetics 32, 314-331) and PCR-RFLP (Damiani et al. 1990, Animal Genetics 21, 107-114; Medrano & Aguilar-Cordova 1990, Animal Biotechnology 1,73-77), allele-specific PCR (=ARMS, ASPCR, PASA; Newton et al. 1989, Nucleic Acids Research 17, 2503-2516; Sakar et al. 1990, Analytical Biochemistry 186, 64-68; David & Deutch 1992, Animal Genetics 23, 425-429), oligonucleotide-ligation assay (=OLA; Beck et al. 2002, J Clinical Mikrobiol 40, 1413-1419), temperature gradient gel electrophoresis (=TGGE, Tee et al. 1992, Animal Genetics 23, 431-435) and analogical procedures belonging to the technical state of the art.

It is suggested to furnish the primers based on the current invention with a marker (fluorescent, radioactive or similar) and to determine the allelic state with a sequencing machine, autoradiography or chemiluminescence. If non-marked primers are utilized, the determination of the allelic state is carried out by illustrating the fragments according to gel electrophoresis through coloring of the nucleic acids, e.g. with ethidiumbromid (Sambrook et al., 1989) or through the silver-coloring procedure (Bassam et al 1991, Analytical Biochemistry 196, 80-83).

Furthermore, it is possible to utilize different high throughput methods for the mutation screening, including the utilization of oligonucleotide arrays (Dong et al 2001, Genome Research 11, 1418-1424), the TaqMan procedure (Ranade et al 2001, Genome research 11, 1262-1268), the fluorescence polarization method, (Chen et al 1999, Genome Research 9, 492-498), mass spectrometric method (MALI-TOF; Sauer et al. 2002, Nucleic Acids Research 30, e22). This enumeration is exemplary and is not to be understood as limited.

The allelic state is hereby to be understood as the existence of a certain nucleotide sequence within the enriched fragment. FIG. 2 exemplifies the nucleotide sequence of four different allelic states of the marker based on the current invention (FIG. 2, alleles 1, 2, 3 and 4).

In the case of sequencing, a comparison with the corresponding nucleotide sequences 1, 2, 3 and 4 in FIG. 2 must be carried out in order to establish the analogical correlations between the allele types 1 to 4. Based on the indicated nucleotide sequences, it is possible for a person familiar with the state of the art to determine the lengths of the fragments through a PCR-RFLP analysis or to conceive oligonucleotides for the detection through allele-specific PCR. Also, the adjustment of the other aforementioned techniques for a mutation screening can be done by the expert.

Particularly preferable is the illustration of the allelic states by means of single-strand conformation polymorphisms (SSCP), as the allelic state can be read directly from the fragment pattern. In addition to the detection of the 4 alleles described here, the procedure also enables the recognition of further mutations which are not described here. For that reason it is also particularly well suited for the analysis of the homologous genome region of other animal species than cattle. In order to reduce the duration of the gel electrophoresis, it is recommended to utilize, instead of the entire sequence, a shorter fragment, e.g. the sequence marked with an arrow in FIG. 1 which is defined by the oligonucleotides based on the current invention.

FIG. 4 shows a schematic illustration of the alleles 1 to 4 of the marker CSN1S1 in the SSCP analysis in a 12% acrylamide/bisacrylamide gel (49:1) with a 1% glycerol additive. The fields 1 to 4 represent the four different separation patterns of the alleles. The migration direction of the molecules in the electrical field from the cathode (−) to the anode (+) is represented by an arrow. The single strands of the alleles show a typical, clearly different separation pattern one from another. Since by means of silver coloring both DNA single strands are illustrated, each allele is characterized by two bands.

4. Selection of organisms which carry the respectively preferable allelic state of the marker based on the current invention. This can be e.g. the allelic state 1 or 4, which differs from allele 2 by the amount of potential binding sites for transcription factors.

### Practical Embodiments

1. Procedure to Classify the Milk Production Traits Through Determination of the Allelic State of the Marker Based on the Current Invention.

Cattle blood is used as source material. The isolation of the genetic material (genomic DNA) is carried out according to the high-salt method of Montgomery & Sise (1990, NZ J Agric Res 33, 437-441).

In order to realize the amplification of the marker by means of PCR reaction the oligonucleotic sequences based on the current invention are utilized as primers:

Primer 1
CSN1S1pro1f (5′ GAA TGA ATG AAC TAG TTA CC 3′)
Primer 2
CSN1S1pro1r (5′ GAA GAA GCA GCA AGC TGG 3′)

In 15 μl, the reaction solutions respectively contain 20-100 ng genomic DNS to be tested, 10 pmol of each oligonucleotide CSN1S1pro1f and CSN1S1pro1r, 0.5 U Taq DNA polymerase (Peqlab Biotechnologie, Erlangen), 50 μM dNTPs in a standard buffer (10 mM Tris-HCl ph 8.8, 50 mM KCl, 1.5 mM MgCl₂). The temperature program (of the thermo cycler, model iCycler of the company Biorad) is selected as follows: 1 min. −93° C. (1×), (40 sec −91° C., 40 sec. 57° C., 40 sec −70° C.) (30×) and 3 min −70° C. (1×). Afterwards, cooling to 4° C. takes place.

Afterwards, to each reaction solution, 25 μl of a formamide denaturation buffer (95% formamid, 0.025% (w/v) bromphenol blue, 0.025% (w/v) xylencyanol FF, 20 mM EDTA) are added respectively, the mixture is heated for 2 min at 93° C., cooled down in ice water and every 4 μl of the mixture are loaded onto a 12% acrylamide/bisacrylamide gel (49:1) with a 1% glycerol additive. The separation is carried out over 20 h at 420V and 10° C. in a vertical electrophoresis system, utilizing the square strip container Penguin P9DS (OWL Scientific, Woburn, USA) with a gel of 0.8 mm thickness and a size of 16×16cm. As running and gel buffer, 0.5× TBE has been utilized. After completion of the electrophoresis, the gels have been colored with silver nitrate according to the protocol of Bassam et al. (1990, Analytical Biochemistry 196, 80-83). The development reaction has been stopped by transferring the gels in an ice-cold 0.04M EDTA solution.

The migration pattern of alleles 1 to 4 is shown schematically in FIG. 3. As by means of silver coloring, both (coding and non-coding DNA line) are colored, two fragments per allele are available respectively.

2. Illustration of the Variability of the Marker CSN1S1 in Various Cattle Breeds

From DNA of 83 cattle of the breeds German American Holstein (6 cattle), German Red cattle (4 cattle), Yellow cattle (7 cattle), German Holstein (18 cattle), Black and White (9 cattle), Jersey (13 cattle), Pinzgauer (20 cattle) and Simbrah (6 cattle) the nucleic acid sequence position 1 to 655, as mentioned in FIG. 1, is amplified with the oligonucleotides, based on the current invention, CSN1S1pro1f (5′ GAA TGA ATG AAC TAG TTA CC 3′) and CSN1S1pro1r (5′ GAA GAA GCA GCA AGC TGG 3′) by means of PCR. The further procedure takes place as described in example 1.

In the breeds examined, the typical separation patterns, as revealed in FIG. 3, appear.

3. Illustration of the Variability of the Marker CSN1S1 Within the German Holstein Breed

From blood samples of 503 cows of the breed German Holstein, DNA is isolated according to the method of Mongomery & Sise (1990, NZ J Agric Res 33, 437-441). The enrichment of the sequence given in FIG. 2 is achieved with the primers based on the current invention, as described above. The illustration of the existing variations is achieved by means of SSCP-Technology. Among the cows examined of this breed, all four alleles are also detectable. The following allele frequencies were determined:

| | |
|---|---|
| Allele 1 - 0,031 | Allele 2 - 0,739 |
| Allele 3 - 0,194 | Allele 4 - 0,036 |

Thus, allele 2 represents the most frequent allele in the German Holstein breed, followed by allele 3 and the two rare alleles 1 and 4.

The genotypes occurred in the frequency 22>23>24>12>33>34. The genotypes 11 and 14 as well as the combination of these two rare alleles (genotype 14) were not found.

4. Genetic Mapping of the Marker CSN1S1

By means of the procedure, based on the current invention, eight half-sib families of the breeds German Holstein (7) and Black and White (1) are genotyped with the marker CSN1S1 and subsequently compared with the results obtained by Thomsen et al. 2000 (J Anim Breed Genet 117, 289-306), who already has genotyped these families for 10 further markers on BTA 6 (microsatellite marker) and has established a linkage map. The genotyping data for CSN1S1 are integrated into this existing data record. Mapping utilizing the BUILD function of the program package CRI-MAP (version 2.4, Green et al. 1990, Documentation of CRI-MAP, Washington School of Medicine, St. Louis, Mo., USA) leads to two possible locations of the CSN1S1 marker: between the markers IL97 and FBN14 or FBN14 and CSN3. The additionally realized FLIPS analysis leads to a definitive mapping of CSN1S1 between the markers FBN14 and CSN3. The total length of the linkage map of BTA6, calculated with these 11 markers, is 161.1 cM. The position of all markers included in the linkage map, and the corresponding indications from the existing linkage maps MARC97 and IBRP97, can be seen in the following table 1. Given are the markers to establish the linkage map of BTA6, the number of informative meioses as well as the positions (cM) on the genetic map calculated with CRI-MAP (the map based on the current invention is referred to as “ADR”) in comparison to the two linkage maps MARC97 and IBRP97. For those markers indicated with “n.a.”, no mapping is available in the respective linkage maps.

TABLE 1

| | | Position (cM) | | |
|---|---|---|---|---|
| Marker | Informative | ADR | MARC97 | IBRP97 |
| ILSTS93 | 193 | 0.0 | 0.0 | 16.0 |
| ILSTS90 | 156 | 28.5 | 11.8 | 0.0 |
| BM1329 | 141 | 56.8 | 35.5 | 45.0 |
| URB16 | 228 | 57.9 | n.a. | 40.0 |
| DIK82 | 356 | 78.5 | n.a. | 67.0 |
| ILSTS097 | 78 | 99.6 | 67.2 | 89.0 |
| FBN14 | 187 | 104.1 | n.a. | n.a. |
| CSN1S1 | 280 | 108.1 | (like CSN3) | (like CSN3) |
| CSN3 | 102 | 113.5 | 82.6 | 103.0 |
| BP7 | 208 | 123.6 | 91.2 | n.a. |
| BMC4203 | 186 | 161.1 | 112.9 | n.a. |

5. Variance Analysis for Estimating the Effects on the milk Production Traits

By means of the procedure based on the current invention a total amount of 729 bulls from 9 half-sib families of the breeds German Holstein and Simmental were genotyped with the marker CSN1S1. The distribution of the genotypes within the 9 half-sib families is shown in table 2.

TABLE 2

| | | CSN1S1 Genotype | | | | | | | |
|---|---|---|---|---|---|---|---|---|---|
| Family | n | 12 | 13 | 14 | 22 | 23 | 24 | 33 | 34 |
| 1 | 19 | — | — | — | 9 | 10 | — | — | — |
| 2 | 108 | 48 | 5 | 5 | 37 | 9 | 4 | — | — |
| 3 | 106 | 4 | — | 3 | 40 | 10 | 37 | — | 12 |
| 4 | 27 | 12 | 3 | 2 | — | 10 | — | — | — |
| 5 | 12 | — | 1 | — | 5 | 5 | — | 1 | — |
| 6 | 27 | — | — | — | 9 | 16 | 1 | 1 | — |
| 7 | 55 | 1 | — | 1 | 22 | 4 | 23 | | 4 |
| 8 | 56 | 4 | 2 | — | 26 | 17 | 3 | 1 | 3 |
| 9 | 319 | 10 | — | — | 250 | 50 | 9 | | — |
| total | 729 | 79 | 11 | 11 | 398 | 131 | 77 | 3 | 19 |

The breeding values of the bulls are centrally estimated by the United Information Systems Animal Production (Vereinigte Informationssysteme Tierhaltung—VIT) in Verden. A total amount of more than 150,000 daughters and their performance data are integrated in the estimation of the breeding values. From all bulls, deregressed breeding values, concerning the milk yield, the protein and fat yield, the protein content (in %) and the fat content (in %), are utilized in the variance component estimation. The deregression of the breeding values is carried out as described by Thomsen et al. (2001, J Anim Breed Genet. 118, 357-370).

The variance component estimation is carried out using the program package SAS. First, as unique fixed effect, the marker CSN1S1 is considered in the model, because other influence factors (e.g. operational effects, milking frequency) are already corrected in the frame of the estimation of the breeding value and the deregression (influence of the sires). The analysis reveals significant effects of the marker CSN1S1 on all studied traits (deregressed breeding values for protein percentage (DRG_PP), milk yield (DRG_MY1), fat yield (DRG_FY1), protein yield (DRG_PY1), fat percentage (DRG_FP)). Table 3 shows the effect of CSN1S1 on deregressed breeding values for milk production traits, indicating also the probability of error (p) for the effects on the individual traits.

TABLE 3

| Trait | Probability of error (p) |
|---|---|
| DRG-PP | <0.0001 |
| DRG_MY1 | 0.0011 |
| DRG_FY1 | 0.0016 |
| DRG_PY1 | 0.0056 |
| DRG_FP | 0.0052 |

The highest significance is calculated for the effect on DRG_PP. As the examined marker CSN1S1 is located directly within the regulatory region of a milk protein gene, this could be an indication of a direct effect. The marker CSN1S1 fulfils the requirements to a functional candidate gene.

The highest breeding value for milk (DRG_MY1) is achieved on average by bulls with the genotype 12, whereas the highest breeding values for protein percentage (DRG_PP) are found within the group with genotype 24. Table 4 shows a compilation of the least square means (LS_means) for the groups with the genotypes 12, 22, 23 and 24. The table displays the LS_means as well as standard errors for the deregressed breeding values for milk yield (DRG_MY1) and protein percentage (DRG_PP) in groups with different CSN1S1 genotypes.

TABLE 4

| CSN1S1 | | LSMEAN ± se | |
|---|---|---|---|
| type | n | DRG_MY1 | DRG_PP |
| 12 | 79 | 198.232 ± 15.700 | −0.00022534 ± 0.00006470 |
| 22 | 398 | 155.341 ± 6.995 | −0.00037495 ± 0.00002921 |
| 23 | 131 | 138.806 ± 12.192 | −0.00038405 ± 0.00005271 |
| 24 | 76 | 112.364 ± 16.007 | 0.00008175 ± 0.00006650 |
| Alle | 684 | 152.353 | −0.000307 |

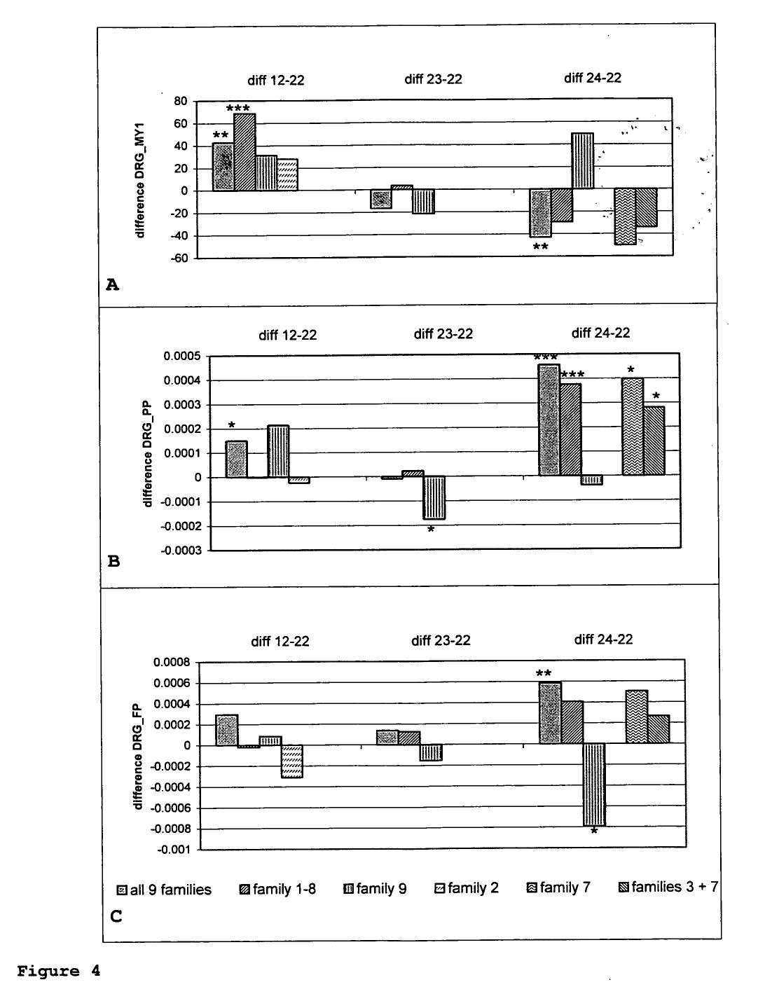

In order to obtain a more exact clarification, the variance analysis is repeated within individual families and groups of families with identical genotypes. Hereby is revealed, that the effect on the milk yield can not be confirmed in all families. In family 9, in which the sires exclusively passed down the allele 2, the only remaining effect is encountered close to the 5% threshold of significance for DRG_PP (p=0.0610). Furthermore, a comparison of the LS_means for the traits DRG_MY1, DRG_PP, DRG_FP is carried out for all groups of genotypes and within each individual family, and it is proved whether the difference of the LS-means between the genotypes 12, 23 and 24 and the most frequent genotype 22 is significant. The results are graphically illustrated in FIG. 5.

1. Genetic marker at the 5′-flanking region of the αS1 casein gene (CSN1S1) characterized by the fact that it contains the nucleotide sequence 1-1061, preferably the nucleotide sequence 1-655 at the 5′-flanking region of the αS1 casein gene.

2. Genetic marker according to patent claim 1 characterized by its amplification by means of PCR reaction either through
Primer 1
CSN1S1pro1f (5′ GAA TGA ATG AAC TAG TTA CC 3′)
Primer 2
CSN1S1pro1r (5′ GAA GAA GCA GCA AGC TGG 3′)
or through
Primer 1
CSN1S1pro1f (5′ GAA TGA ATG AAC TAG TTA CC 3′)
Primer 3
CSN1S1pro2r (5′ CCT TGA AAT ATT CTA CCA G 3′)

3. Genetic marker according to patent claim 1 characterized by its variability within milk breeds.

4. Genetic marker according to patent claim 1 characterized by its utilization in order to determine the allelic state at the 5′-flanking region of the αS1 casein gene.

5. Procedure to determine the allelic state of the 5′-flanking region of the αs1 casein gene, characterized by the following steps:
a) provision of the source material of the organism to be examined
b) isolation of the genetic material
c) targeted isolation or enrichment of the marker fragment at the 5′ region of the αs1 casein gene or of a sequence, which contains portions of the marker sequence, preferably the fragment 1 to 655 of the marker sequence out of the αs1 casein gene
d) Proof of the allelic state in the isolated or enriched sequence fragment of the marker fragment of the αs1 casein gene.

6. Procedure according to patent claim 5 characterized by the utilization of source material coming from an animal, particularly a mammal, in particular a bovine, a sheep or a goat, including breed animals and embryos of these species.

7. Procedure according to patent claim 5 characterized by the utilization of blood, leukocytes, tissue including biopsy material, milk, sperm, hair, individual cells including cell material from embryos, a bacteria culture or isolated chromosomes as source material.

8. Procedure according to patent claim 5 characterized by the utilization of source material coming from a genetically modified organism (GMO) which contains the marker fragment of the αs1 casein gene.

9. Procedure according to patent claim 5 characterized by the utilization of genetic material containing genomic DNA or RNA from animals, plasmid DNA from bacteria, from artificial chromosomes such as BACs and YACs.

10. Procedure according to patent claim 5 characterized by achieving the enrichment of the marker segment of the αs1 casein gene by means of polymerase chain-reaction.

11. Procedure according to patent claim 5 characterized by the enrichment of the marker segment of the αs1 casein gene by means of polymerase chain-reaction with the oligonucleotides
Primer 1
CSN1S1pro1f (5′ GAA TGA ATG AAC TAG TTA CC 3′)
Primer 2
CSN1S1pro1r (5′ GAA GAA GCA GCA AGC TGG 3′)
Primer 3
CSN1S1pro2r (5′ CCT TGA AAT ATT CTA CCA G 3′)
as primers, whereby the following combinations are selected: primer 1 with primer 2 and primer 2 with primer 3.

12. Procedure according to patent claim 5 characterized by the determination the allelic state by means of SSCP, RFLP, OLA, TGGE, ASPCR, PCR-ELISA, microarray method or through nucleic acid sequencing.

13. Procedure according to patent claim 5 characterized by detection of one or more of the allelic states of the marker sequence of the αs1 casein gene.

14. Utilization of the procedure according to claim 5 in order to examine the animals' milk production traits, independently of age and lactation.

15. Utilization of the procedure according to claim 5 in order to select organisms which carry a certain allelic state or a certain genotype of the marker sequence of the αs1 casein gene or a portion thereof.

16. Utilization of the procedure according to claim 5 in breeding programs, particularly for a marker-supported selection.

17. Utilization of the procedure according to claim 5 for the selection of increased milk protein yields.

18. Utilization of a marker according to patent claim 1 for genome analysis, in particular to carry out a genetic mapping and/or a linkage analysis.

19. Utilization of a marker according to patent claim 1 to create expression vectors.

20. Utilization of a marker according to patent claim 1 to produce transgenic animals.

21. Testkit, containing oligonucleotides to enrich a segment of the marker sequence of the as I casein gene, preferably the primer 1 CSN1S1pro1f (5′ GAA TGA ATG AAC TAG TTA CC 3′), primer 2 CSN1S1pro1r (5′ GAA GAA GCA GCA AGC TGG 3′) and primer 3 CSN1S1pro2r (5′ CCT TGA AAT ATT CTA CCA G 3′) as well as reference probes for one or various sequences of the marker sequence of the αs1 casein gene and the alleles thereof.


SEQUENCE LISTING (8 sequences)

SEQ ID NO: 1 - DNA, 18 bp, Bos spec.
  Primer 2 (1)..(18): 18 basepair, single stranded nucleic acid (linear)
gaagaagcagcaagctgg

SEQ ID NO: 2 - DNA, 19 bp, Bos spec.
  Primer 3 (1)..(19): 19 basepair, single stranded nucleic acid (linear)
ccttgaaatattctaccag

SEQ ID NO: 3 - DNA, 1061 bp, Bos taurus
  alpha-S1Kaseingen (1)..(1061): start Exon 1 at position 620
  ref <301>: AUTHORS: Koczan Dirk, Hobom Gerd, Seyfert Hans-Martin
  ref <302>: TITLE: Genomic organization of the bovine alpha S1-casein gene
  ref <303>: JOURNAL: Nucleic acids research
  ref <304>: VOLUME: 19
  ref <305>: ISSUE: 20
  ref <306>: PAGES: 5591
  ref <307>: DATE: 1991-09-24
  ref <308>: DATABASE ACCESSION NUMBER: X59856
  ref <309>: DATABASE ENTRY DATE: 1991-07-18
  ref <313>: RELEVANT RESIDUES: (1)..(1061)
  ref <308>: DATABASE ACCESSION NUMBER: EMBL X59856
  ref <309>: DATABASE ENTRY DATE: 1991-07-18
  ref <313>: RELEVANT RESIDUES: (1)..(1061)
gaatgaatgaactagttaccacaactagtacacccaaaatgaacaaaaaatagcttggtggtataattaaaatgccaccaaaatttatacaataattatattttctttttgcaggaaaaagattagaccacatataatgtaacttatttcacaaggtaaataattataataaataatatggattaactgagttttaaaaggtgaaataaataatgaattcttctcatggtcttgtatgttaataaaaattgaaaaattttgaagaccccattttgtcccaagaatttcatttacaggtattgaatttttcaaaggttacaaaggaaattttattgatataataaatgcatgttctcataataaccataaatctagggttttgttggggtttttttttgtttgttaatttagaacaatgccattccatttcctgtataatgagtcacttctttgttgtaaactctccttagaatttcttgggagaggaactgaacagaacattgatttcctatgtgagagaattcttagaatttaaataaacctgttggttaaactgaaaccacaaaattagcattttactaatcagtaggtttaaatagcttggaagcaaaagtctgccatcaccttgatcatcaacccagcttgctgcttcttcccagtcttgggttcaaggtattatgtatacatataacaaaatttctatgattttcctctgtctcatctttcattcttcactaatacgcagttgtaacttttctatgtgattgcaagtattggtactttcctatgatatactgttagcttaaaaatatatttgcaaatgttgatactatctatctcagagctataggtgaaaaattaaatacttttataaagaccaaattgatcatttttaaacgaaattcttatatactgaaaatgtagatacataacttcagtatagatttatggtaaaataatttgaatcatttttgtcaaattctgtaaaaagttgtcatacagaataatttataatatttttgttttcatagaaataacatttctggtagaatatttcaagg

SEQ ID NO: 4 - DNA, 652 bp, Bos taurus
  CSN1S1-gene, 5`flanking region from position 616 and Exon 1 at position 617 (1)..(652): Mutation/SNP position 83 (A to G), position 98 (A to G), position 298 (A to C), position 442 (A to G; change/loss of  YY1- and AP1 -bindingsite), position 541 (G to A); deletion TT between position 389 and 394 compaired with Allel2
gaatgaatgaactagttaccacaactagtacacccaaaatgaacaaaaaatagcttggtggtataattaaaatgccaccaaagtttatacaataattgtattttctttttgcaggaaaaagattagaccacatataatgtaacttatttcacaaggtaaataattataataaataatatggattaactgagttttaaaaggtgaaataaataatgaattcttctcatggtcttgtatgttaataaaaattgaaaaattttgaagaccccattttgtcccaagaatttcctttacaggtattgaatttttcaaaggttacaaaggaaattttattgatataataaatgcatgttctcataataaccataaatctagggttttgttggggttttttgtttgttaatttagaacaatgccattccatttcctgtataatgagtcgcttctttgttgtaaactctccttagaatttcttgggagaggaactgaacagaacattgatttcctatgtgagagaattcttagaatttaaataaacctattggttaaactgaaaccacaaaattagcattttactaatcagtaggtttaaatagcttggaagcaaaagtctgccatcaccttgatcatcaacccagcttgctgctttctt

SEQ ID NO: 5 - DNA, 654 bp, Bos taurus
  CSN1S1-gene, 5`flanking region and Exon 1 (1)..(654): Bindingsite for transcriptionfactor AP-1 at position 438 to 445 Bindingsite for transcriptionfactor YY-1 at position 443 to 448
gaatgaatgaactagttaccacaactagtacacccaaaatgaacaaaaaatagcttggtggtataattaaaatgccaccaaaatttatacaataattatattttctttttgcaggaaaaagattagaccacatataatgtaacttatttcacaaggtaaataattataataaataatatggattaactgagttttaaaaggtgaaataaataatgaattcttctcatggtcttgtatgttaataaaaattgaaaaattttgaagaccccattttgtcccaagaatttcatttacaggtattgaatttttcaaaggttacaaaggaaattttattgatataataaatgcatgttctcataataaccataaatctagggttttgttggggttttttttgtttgttaatttagaacaatgccattccatttcctgtataatgagtcacttctttgttgtaaactctccttagaatttcttgggagaggaactgaacagaacattgatttcctatgtgagagaattcttagaatttaaataaacctgttggttaaactgaaaccacaaaattagcattttactaatcagtaggtttaaatagcttggaagcaaaagtctgccatcaccttgatcatcaacccagcttgctgctttctt

SEQ ID NO: 6 - DNA, 650 bp, Bos taurus
  CSN1S1-gene, 5`flanking region (1)..(650): Bindingsite for transcriptionfactor AP-1 at position 434 to 441 Bindingsite for transcriptionfactor YY-1 at position 439 to 444 deletion G and TTT between 390 and 396 compaired with  Allel 2
gaatgaatgaactagttaccacaactagtacacccaaaatgaacaaaaaatagcttggtggtataattaaaatgccaccaaaatttatacaataattatattttctttttgcaggaaaaagattagaccacatataatgtaacttatttcacaaggtaaataattataataaataatatggattaactgagttttaaaaggtgaaataaataatgaattcttctcatggtcttgtatgttaataaaaattgaaaaattttgaagaccccattttgtcccaagaatttcatttacaggtattgaatttttcaaaggttacaaaggaaattttattgatataataaatgcatgttctcataataaccataaatctagggttttgttggggttttttttgttaatttagaacaatgccattccatttcctgtataatgagtcacttctttgttgtaaactctccttagaatttcttgggagaggaactgaacagaacattgatttcctatgtgagagaattcttagaatttaaataaacctgttggttaaactgaaaccacaaaattagcattttactaatcagtaggtttaaatagcttggaagcaaaagtctgccatcaccttgatcatcaacccagcttgctgctttctt

SEQ ID NO: 7 - DNA, 650 bp, Bos taurus
  CSN1S1-gene, 5`flanking region (1)..(650): Bindingsite for transcriptionfactors: AP-1 at position 434 to 441,  ABF1 at position 469 to 483, YY-1 at position 439 to 444; mutation (SNP) at position 480 (G to C), developing a ABF1-bindingsite; deletion G and TTT between position 390 and 396 compaired with Al lel 2
gaatgaatgaactagttaccacaactagtacacccaaaatgaacaaaaaatagcttggtggtataattaaaatgccaccaaaatttatacaataattatattttctttttgcaggaaaaagattagaccacatataatgtaacttatttcacaaggtaaataattataataaataatatggattaactgagttttaaaaggtgaaataaataatgaattcttctcatggtcttgtatgttaataaaaattgaaaaattttgaagaccccattttgtcccaagaatttcatttacaggtattgaatttttcaaaggttacaaaggaaattttattgatataataaatgcatgttctcataataaccataaatctagggttttgttggggttttttttgttaatttagaacaatgccattccatttcctgtataatgagtcacttctttgttgtaaactctccttagaatttcttgggagacgaactgaacagaacattgatttcctatgtgagagaattcttagaatttaaataaacctgttggttaaactgaaaccacaaaattagcattttactaatcagtaggtttaaatagcttggaagcaaaagtctgccatcaccttgatcatcaacccagcttgctgctttctt

SEQ ID NO: 8 - DNA, 20 bp, Bos spec.
  Primer1 (1)..(20): 20 basepair, single stranded nucleic acid (linear)
gaatgaatgaactagttacc